(19) United States
(12) Patent Application Publication
(10) Pub. No.: US 2020/0397314 A1
(43) Pub. Date: Dec. 24, 2020

(54) CUTANEOUS SYSTEM FOR MONITORING AN INDIVIDUAL

(71) Applicant: DEVINNOVA, Montpellier (FR)

(72) Inventor: Fabrice VAUSSENAT, Montpellier (FR)

(21) Appl. No.: 16/755,529

(22) PCT Filed: Oct. 12, 2018
(86) PCT No.: PCT/EP2018/077949

§ 371 Date: Apr. 10, 2020

(30) Foreign Application Priority Data
Oct. 13, 2017 (FR) 1759631

(51) Int. Cl. A61B 5/0205; A61B 5/0408; A61B 5/113; A61B 5/1455; A61B 5/00

(52) U.S. Cl. CPC A61B 5/02055; A61B 5/04085; A61B 5/1135; A61B 5/14552; A61B 5/442; A61B 5/6833; A61B 2562/0271; A61B 2560/045; A61B 2562/0219; A61B 2562/0247; A61B 2562/166; A61B 2562/227; A61B 5/0022

(57) ABSTRACT

The invention relates to a cutaneous patch for monitoring the heart rate of an individual, as well as to a monitoring system comprising such a patch, and to an assembly that comprises a system of this type.

### GENERAL TECHNICAL FIELD

The invention relates to a cutaneous patch intended to collect cardiac signals as well as other physiological parameters of an individual, for the purpose of obtaining parameters characteristic of the cardiac activity of the individual patient. These parameters allow real time monitoring of the cardiac activity of the individual in an ambulatory setting.

The invention also relates to a monitoring system for the cardiac activity of an individual comprising a patch of this type, the monitoring being able to be carried out remotely.

### PRIOR ART

The monitoring of heart rate in an ambulatory setting is used in numerous cases: suspected heart disease, effect of certain treatments, etc.

Several monitoring devices are known for accomplishing this. Those most used are devices of the Holter ECG type, which allow recording the ECG of the individual.

One problem is that this type of device is not practical because it is bulky.

Another problem is that it only allows acquiring a certain type of signal and does not allow real-time follow-up: the individual must wear the device for 24 hours, and have the signals analyzed by a practitioner.

Yet another problem is that, taking into account the limited number of different signals acquired, the characteristic parameters of the cardiac activity of the individual obtained are limited.

Consequently, there exists a need to have a cardiac activity monitoring device which can operate in real time, which is practical for an individual to wear and which can contribute to obtaining several parameters characteristic of the cardiac activity of the individual.

### PRESENTATION OF THE INVENTION

The invention responds the above need and proposes, according to a first aspect, a cutaneous patch for monitoring heart rate intended to be worn by an individual, the patch comprising, successively
a connection layer comprising a first adhesive face intended to be in contact with the skin of the individual and a second face;
a flexible printed circuit comprising
   a first face supporting a plurality of sensors, said first face of said circuit being in contact with the second face of the connection layer and said sensors comprising:
      three electrodes configured to acquire signals representing the cardiac activity of the individual, said electrodes being positioned in an equilateral triangle,
      a pressure sensor configured to measure the respiratory frequency of the individual
      a connector to which an electronic module is intended to be connected; said module being configured to control the sensors in order to acquire physiological parameters of the individual and, from said acquired parameters, to determine, in situ, characteristic parameters of the cardiac activity of the individual;
   a second face, opposite the first face and supporting contacts coming from the sensors;
a shell covering the connection layer and the flexible printed circuit, said shell comprising a lower portion in contact with the second face of the flexible printed circuit and an upper portion intended to be in the open, said lower portion comprising a housing intended to house the electronic module;
   the connection layer comprising a strip having a shape suitable for covering the housing of the shell, said strip allowing opening or closing the housing in order to allow the insertion or the withdrawal of the electronic module, the shell and the strip together contributing to ensure the sealing of the housing so as, on the one hand, to resist water and, on the other hand, to maintain a constant pressure in the housing.

The invention is advantageously completed by the following features, taken alone or in any one of their technically possible combinations.

The printed circuit further supports:
two temperature sensors configured to measure the skin temperature of the individual;
an infrared signal transmitter/receiver configured to measure the oxygen saturation of the individual;
un accelerometer configured to determine the posture of the individual.

The printed circuit further supports a piezoelectric sensor configured to measure the density of tissues by elastography.

The electrodes are positioned equilaterally and are spaced two by two at a distance comprised between 65 mm and 82 mm, typically 78 mm.

The infrared transmitter/receiver is configured to emit a signal having a wavelength comprised between 1420 mm and 1800 mm, typically between 1450 mm and 1700 mm.

The infrared transmitter/receiver is configured to emit a signal toward the skin of the individual, which is refracted by the skin at an angle comprised between 25° and 65°, typically between 30° and 60°.

The lower portion of the shell comprises a space provided in said lower portion so as to house a battery such as a button type cell.

The upper portion of the shell comprises a protection layer above the space intended to house the battery, said protection layer being configured to block the electromagnetic waves coming from the battery during operation of the patch, said protection layer being preferably of polycarbonate.

The patch comprises hydrogel pads positioned on each of the electrodes, the electrodes being in contact with the skin of the individual by means of said pads.

The layer comprises three cutouts of the size of the electrodes so as to allow the contact of the electrodes with the skin of the individual.

The invention also relates to a cutaneous monitoring system comprising a patch according to the invention and an electronic module electrically connected to the flexible printed circuit, said electronic module being configured to control the sensors and, based on the information from the sensors, to determine characteristic parameters of the cardiac activity of the individual.

The invention also relates to a cutaneous monitoring assembly comprising a monitoring system according to the preceding claim, a mobile terminal, the monitoring system being in wireless connection with the mobile terminal.

The advantages of the invention are numerous.

The structure of the patch, which comprises few layers, is particularly advantageous in that its manufacture is simple and easily repeatable.

Using the patch, it is possible to measure the electrical signals of the heart using three derivations, thus allowing having morphological information (right heart, left heart) on the heart and specifying the origin of the rhythm disorder.

The infrared (IR) measurement by reflection allows measuring the heat rate, thus ensuring the reliability of measurement from the ECG but also measuring the variation of the SPO2.

The measurement of the temperature by two temperature sensors of the thermistor type also ensures differential measurement of temperature. This measurement allows us to follow the slope of the temperature in order to know whether the temperature of the patient is dropping or increasing.

The pressure sensor housed in a sealed chamber at constant pressure allows accurate measurement of the pressure variation coming from costal movement with regard to the respirator frequency deduced from the ECG.

Moreover, the measurement of pressure by a dedicated sensor allows having a more accurate measurement than when the pressure is deduced from the ECG.

The measurement of density allows the evaluation of ischemic necroses and proposes an evaluation of unknown antecedents.

The information combined in the specific algorithms allows measuring indicators (physiomarkers), thus allowing the precocious detection of infarction, of rhythm disorder, of respiratory distress.

The lightness of the patch, as well as the absence of wires, allows the user to have complete freedom with regard to his activity. Thus the problems of disconnection of the wires on the electrodes, the limit on usage under stress conditions are limitations which are lifted and allow optimization of support.

In fact, the use of the patch under stress is possible, because it integrates filtration of artefacts linked to myoelectric noise.

The invention allows real-time monitoring of the cardiac activity of an individual. In fact, the presence of an electronic module controlling the sensors and performing necessary processing for obtaining this information in real time.

The patch comprising several sensors can in particular be used for the detection of cardiac disorders: bradycardia, tachycardia, atrial (or auricular) fibrillation, ventricular fibrillation, ventricular arrhythmias, extrasystoles (atrial or ventricular), cardiac conduction disorders, infarction.

### PRESENTATION OF THE FIGURES

Other features, aims and advantages of the invention will be revealed by the description that follows, which is purely illustrative and not limiting, and which must be read with reference to the appended drawings in which:

FIG. 1 illustrates a monitoring assembly according to one embodiment of the invention;

FIG. 2 illustrates a front view of a cutaneous patch of a cutaneous monitoring system according to one embodiment of the invention;

FIG. 3 illustrates an exploded view of a cutaneous monitoring system according to one embodiment of the invention;

FIG. 4 illustrates a view of a printed circuit of a cutaneous monitoring system according to the invention;

FIG. 5 illustrates schematically an electronic module of a cutaneous monitoring system according to the invention.

In all the figures, similar elements carry identical reference symbols.

### DETAILED DESCRIPTION OF THE INVENTION

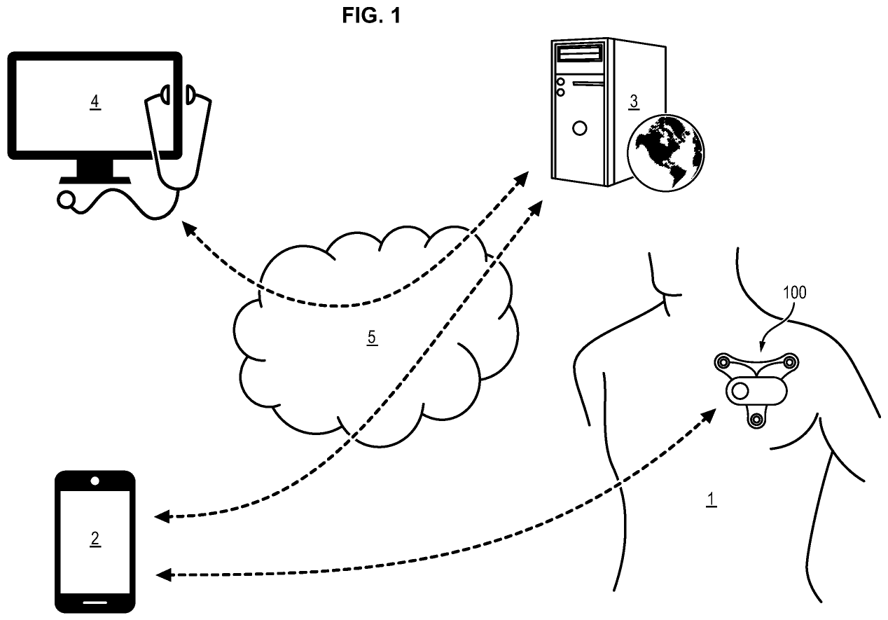

FIG. 1 illustrate an individual 1 wearing a cutaneous monitoring system 100 comprising a patch 10 according to one embodiment.

The system 100 is intended to acquire, besides cardiac signals (called ECG signals), several signals characteristic of physiological parameters of the individual: skin temperature, posture, oxygen saturation, muscular mass density.

These signals allow obtaining parameters characteristic of the cardiac activity of the individual.

The system 100 can advantageously be connected to a mobile terminal 2 (smartphone or tablet for example) via a wireless connection of the Bluetooth type.

The mobile terminal 2 carries on board a mobile application which allows recovering the data from the system 100 to process them, display them or even transmit them to a remote server 3.

For this reason, the mobile terminal 2 can be connected to the remote server 3 via a mobile network 5 of the Internet type. The connection to this server is preferably secured. This server 3 allows remote storage of data from the system 100 passing through the mobile terminal 2. This remote server 3 is connected via the mobile network 5 to a remote terminal 4 comprising an interface (not shown) allowing a user, such as a physician, to gain access to the data acquired as well as to the characteristic parameters of the cardiac activity of the individual. Via this terminal 4, a physician or any other authorized person can possibly receive alerts depending on the parameters characteristic of cardiac activity obtained. This is particularly advantageous when the aim is to remotely monitor individuals at risk.

Patch 10

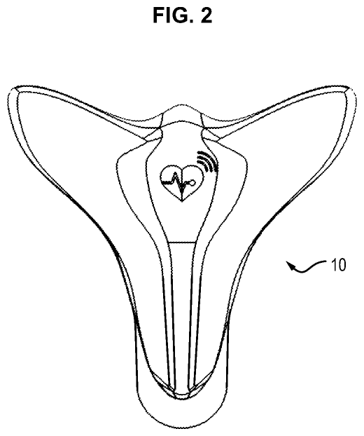

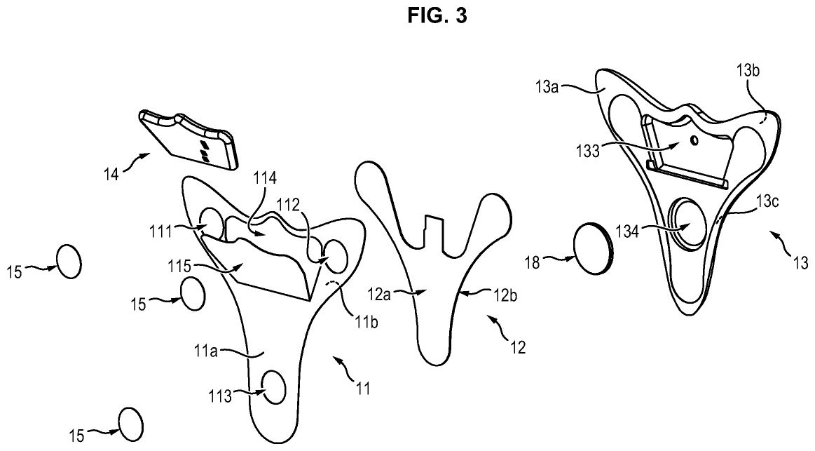

As mentioned, the system 100 is intended to be worn by an individual and comprises a cutaneous patch 10 (visible in FIG. 2) consisting of several layers (visible in FIG. 3) and of an electronic module 14 supplied with power by a battery 18 such as a button type cell.

The patch 10 advantageously comprises the following successive stack:

a connection layer 11 comprising a first adhesive face 11a in contact with the skin and a second adhesive face 11b;

a flexible printed circuit 12;

a shell 13 covering the connection layer and the printed circuit 12.

A skin connection layer 11 ensures the connection of the patch 10 with the skin of the individual. The first face 11a is therefore adhesive and supports a hypoallergenic adhesive to prevent possible allergic reactions. The first adhesive face 11a is preferably protected by a removable sheet (not shown) which is removed before the application of the patch 10 on the skin.

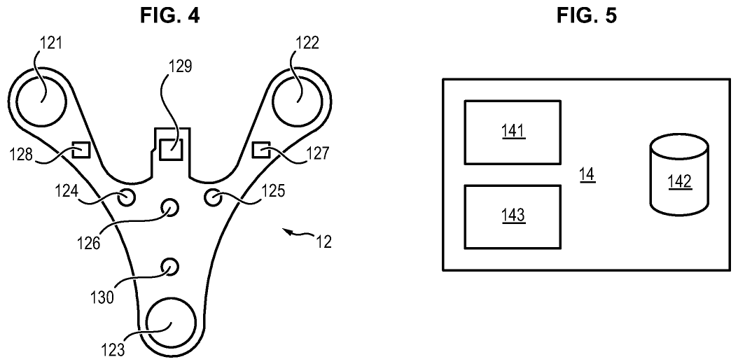

The second, nonadhesive face 11b (the one not intended to be in contact with the skin) supports a printed circuit 12 (illustrated in detail in FIG. 4), itself supporting a certain number of sensors which allow measuring a certain number of signals representative of the cardiac activity of the individual.

The flexible printed circuit 12 comprises a first face 12a supporting several sensors. The first face of the printed circuit 12 is in contact with the second face 11b of the connection layer 11. The printed circuit 12 comprises three electrodes 121, 122, 123 in contact with the skin of the individual by means of three cutout zones 111, 112, 113 in the connection layer 11. Complementarily, hydrogel cushions 15 (one for each electrode) allow increasing the contact surface of the electrodes with the skin of the individual and absorbing the water from the pores of the skin.

The printed circuit 12 comprises a connector 129 on which is connected an electronic module 14 connected electrically to the printed circuit 12. In this manner, it is possible to withdraw the electronic module 14 to reuse it with another patch or to perform updates.

The connector 129 is accessible from the first, adhesive face 11a of the connection layer via an opening 114 re-closable using a strip 115. This strip 115 facilitates access to the electronic module 14 and allows ensuring the sealing of the zone in which the electronic module 14 is located.

The shell 13 comprises a lower portion 13a covering the second face 12a of the printed circuit 12. This lower portion 13a comprises a first housing 133 in which the electronic module 14 is positioned. The lower portion 13a also comprises a second housing 134 in which is housed a battery 18 (a cell for example). The first housing 133 is therefore accessible from the first, adhesive face 11a of the connection layer 11 via the opening 114 and the strip 115.

The shell 13 also comprises an upper portion 13b which is in the open and which ensures the sealing of the patch. This upper portion 13b is preferably of polyurethane. Thus the shell 13 is a layer for protection from the elements constitutive of the patch 10.

Advantageously, the portion covering the power pack 18 comprises a canopy of polycarbonate allowing blocking electromagnetic emissions from the battery which can perturb the printed circuit 12 and the electronic module 14. This canopy also allows absorbing the humidity emitted by sweat when the patch is worn and absorbing the heat emitted by the battery.

In this manner, the patch can be worn 24h out of 24h for a duration of approximately seven days.

Printed Circuit 12

The printed circuit 12 is illustrated in FIG. 4 and comprises several sensors among those described hereafter (the simultaneous presence of the sensors is not limiting).

It consists of a printed circuit 12 of flexible material so as to favor its integration within the patch 10 for the purpose of its application to the skin of the individual, where contact with the skin must be optimal.

As already mentioned, three electrodes 121, 122, 123 are positioned equilaterally (at a 60° angle). The electrodes allow acquiring ECG signals from the heart of the individual. The electrodes are preferably spaced two by two by a distance comprised between 65 mm and 82 mm, typically 78 mm. Such positioning allows limiting the overlapping effects of the electrical signals of the heart and ensure a “right heart,” “left heart” measurement. These “right heart,” “left heart” measurements allow obtaining morphological information from the heart. The electrodes are advantageously covered with a deposit of silver chloride so as to reduce the risks of oxidation.

Two temperature sensors 124, 125 positioned 10 mm from the electrodes at an angle of 45° relative to the center of the electrode, allow measuring the skin temperature of the individual at two different locations. In this manner, a temperature differential is measured. The differential corresponds to the slope which allow indicating whether the temperature is increasing or dropping. The measurement is the image of the derivation of the temperature, which constitutes an essential element in following the evolution of vital parameters.

An infrared signal transmitter/receiver 126 allows measuring the oxygen saturation of the individual. In particular, the transmitter emits an infrared signal in a wavelength comprised between 1420 mm and 1800 mm, typically between 1450 mm and 1700 mm. In addition, the signal emitted is such at it has, at the air/skin interface, a reflection angle comprised between 25° and 65°, typically between 30° and 60°. The angle of reflection provided for a reduction in variability of the saturation by averaging the values of the values measured on the diffusion surface.

A six-axis accelerometer 127 allows measuring the posture of the individual, the speed of the individual, the distance traveled by the individual. These measurements from the accelerometer contribute to evaluating the activity of the patient.

A pressure sensor 128 allows measuring the respiratory frequency of the individual. A sensor 128 of this type allow having a better measurement of the respiratory frequency than when it is deduced from the ECG signals. To allow accurate measurement of the respiratory frequency by this sensor, the housing 133 of the electronic module 14 is at constant pressure thanks to the shell 13 and the strip 115.

A piezoelectric sensor 130 allows measuring the density of the tissues by elastography. In fact, the piezoelectric sensor 130 emits a mechanical wave, and retrieves it by echo. The ratio of the amplitudes of the emitted and received wave allows deducing the density of the tissues. A measurement of this type is useful for subsequently detecting possible cellular necroses.

Electronic Module 14

The electronic module 14, illustrated schematically in FIG. 5, is configured to control the sensors and, based on information from the sensors, to determine characteristic parameters of the cardiac activity of the individual. Advantageously, all the characteristic parameters are obtained in situ in the assembly 100.

In particular, the electronic module 14 comprises a processor 141 configured to implement various processing of the signals from the sensors, a memory 142 configured to store the signals acquired and a wireless communication interface 143 configured to be in communication with the terminal 2 or the server 3 (see FIG. 1).

The processor 141 is in particular configured to control the acquisition of electrical signals from the heart by means of the three electrodes (three derivations). These three derivations I, II and III give an indication on the origin of the rhythm anomaly. The acquisition is preferably performed at the frequency of 400 Hz on each of the electrodes, hence 1200 Hz total, considering the three electrodes.

The signals acquired for each of the electrodes are advantageously filtered by means of two analog filters (one high-pass filter and one low-pass filter) and by means of a digital filter of the wavelet type. The wavelets used obtain their references from the Meyer coefficients, but have undergone modifications in order to improve the detection of each of the peaks and intervals. Wavelet filtration allows extracting the particular elements of the signals acquired. In particular, it allows extracting the P, Q, R, S, T and U waves from the ECG signals acquired by the electrodes.

Based on these waves, the following intervals are advantageously calculated: interval PR, segment PR, interval QRQ, segment ST, interval ST, interval RR.

Finally, these intervals, possibly coupled to one or more pieces of information from the other sensors, allow obtaining the characteristic parameters of the cardiac activity of the individual.

1. A cutaneous patch for monitoring heart rate intended to be worn by an individual, the patch comprising, successively
a connection layer comprising a first face intended to be in contact with the skin of the individual and a second face;
a flexible printed circuit comprising
a first face supporting a plurality of sensors, said first face of said circuit being in contact with the second face of the connection layer and said sensors comprising:
three electrodes configured to acquire signals representing the cardiac activity of the individual, said electrodes being positioned in an equilateral triangle,
a pressure sensor configured to measure the respiratory frequency of the individual
a connector to which an electronic module is intended to be connected; said module being configured to control the sensors in order to acquire physiological parameters of the individual and, from said acquired parameters, to determine, in situ, characteristic parameters of the cardiac activity of the individual;
a second face opposite the first face and supporting contacts coming from the sensors;
a shell covering the connection layer and the flexible printed circuit, said shell comprising a lower portion in contact with the second face of the flexible printed circuit and an upper portion intended to be in the open, said lower portion comprising a housing intended to house the electronic module;
the connection layer comprising a strip having a shape suitable for covering the housing of the shell, said strip allowing opening or closing the housing in order to allow the insertion or the withdrawal of the electronic module, the shell and the strip together contributing to ensure the sealing of the housing so as, on the one hand, to resist water and, on the other hand, to maintain a constant pressure in the housing.

2. The patch according to claim 1, wherein the printed circuit further supports:
two temperature sensors configured to measure the skin temperature of the individual;
an infrared signal transmitter/receiver configured to measure the oxygen saturation of the individual;
an accelerometer configured to determine the posture of the individual;

3. The patch according to claim 1, wherein the printed circuit further supports a piezoelectric sensor configured to measure the density of tissues by elastography.

4. The patch according to claim 1, wherein the electrodes are positioned equilaterally and are spaced two by two at a distance comprised between 65 mm and 82 mm, typically 78 mm.

5. The patch according to claim 1, wherein the infrared transmitter/receiver is configured to emit a signal having a wavelength comprised between 1420 mm and 1800 mm, typically between 1450 mm and 1700 mm.

6. The patch according to claim 1, wherein the infrared transmitter/receiver is configured to emit a signal toward the skin of the individual which is refracted by the skin at an angle comprised between 25° and 65°, typically between 30° and 60°.

7. The patch according to claim 1, wherein the lower portion of the shell comprises a space provided in said lower portion so as to house a battery, such as a button type cell.

8. The patch according to claim 7, wherein the upper portion of the shell comprises a protection layer above the space intended to house the battery, said protection layer being configured to block the electromagnetic waves coming from the battery during operation of the patch, said protection layer being preferably of polycarbonate.

9. The patch according to claim 1, comprising hydrogel pads positioned on each of the electrodes, the electrodes being in contact with the skin of the individual by means of said pads.

10. The patch according to claim 1, wherein the layer comprises three cutouts of the size of the electrodes so as to allow the contact of the electrodes with the skin of the individual.

11. A cutaneous monitoring system comprising a patch according to claim 1 and an electronic module electrically connected to the flexible printed circuit, said electronic module being configured to control the sensors and, based on the information from the sensors, to determine characteristic parameters of the cardiac activity of the individual.

12. A cutaneous monitoring assembly comprising a monitoring system according to claim 11, a mobile terminal, the monitoring system being in wireless connection with the mobile terminal.
